(19) United States
(12) Patent Application Publication
(10) Pub. No.: US 2022/0142530 A1
(43) Pub. Date: May 12, 2022

(54) SYSTEM AND METHOD FOR IMAGE ANALYSIS OF OXIMETRY BURDEN AS A MEASURE OF CARDIOVASCULAR RISK

(71) Applicant: SOMNOLOGY, INC., Redwood City, CA (US)

(72) Inventors: Melissa Susann LIM, San Francisco, CA (US); Charles Lee HAMILTON, Fort Bragg, CA (US); Patrick Joseph YAM, Portola Valley, CA (US); Brian Ko YAM, Portola Valley, CA (US); Minqiao JIN, Stanford, CA (US)

(21) Appl. No.: 17/522,618

(22) Filed: Nov. 9, 2021

Related U.S. Application Data
(60) Provisional application No. 63/112,041, filed on Nov. 10, 2020.

(51) Int. Cl. A61B 5/1455; A61B 5/00

(52) U.S. Cl. CPC A61B 5/14551; A61B 5/7275; A61B 5/743

(57) ABSTRACT

A system and method for image analysis of oximetry burden as a measure of cardiovascular risk are disclosed. A particular embodiment includes: using a data processor to receive a digitized representation of an oximetry graph of a patient; detecting a level of arterial oxygen saturation (SpO2) in the blood of the patient over a given time period based on the digitized representation of the oximetry graph of the patient; and determining a Hypoxemia Burden Index (HBI) for the patient based on a percentage of time over a unit of measure parameter that the levels of SpO2 in the blood of the patient are below a hypoxemia burden threshold.

### PRIORITY PATENT APPLICATION

This non-provisional patent application draws priority from U.S. provisional patent application Ser. No. 63/112,041; filed Nov. 10, 2020. This present non-provisional patent application draws priority from the referenced patent application. The entire disclosure of the referenced patent application is considered part of the disclosure of the present application and is hereby incorporated by reference herein in its entirety.

### TECHNICAL FIELD

This patent application relates to the analysis and quantification of hypoxemia burden and cardiovascular risk in a patient, the clinical study and treatment of sleep disorders and irregularities, and computer-implemented software, according to one embodiment, and more specifically to a system and method for image analysis of oximetry burden as a measure of cardiovascular risk.

### COPYRIGHT

A portion of the disclosure of this patent document contains material that is subject to copyright protection. The copyright owner has no objection to the facsimile reproduction of the patent document or the patent disclosure, as it appears in the Patent and Trademark Office patent files or records, but otherwise reserves all copyright rights whatsoever. The following notice applies to the disclosure herein, the software and data as described below, and to the drawings that form a part of this document: Copyright 2012-2021 Somnology, Inc., All Rights Reserved.

### BACKGROUND

Sleep disorders are extremely common in the general population, with estimates of 50 million to 70 million people suffering from sleep related disorders, including roughly 15 million adults in the U.S. with persistent insomnia, and another 24 million adults and children suffering from obstructive sleep apnea. The current model of care is for people to seek consultation with their primary care doctor first about their sleep issues. From there, people are referred for sleep studies, sleep specialists, or given medications. Treatment is often delayed, involves potentially habit-forming if medications, or is too expensive to pursue. Moreover, it is increasingly recognized that the traditional apnea hypopnea index (AHI) in sleep medicine, although the current measure of sleep apnea severity, is a weak predictor of patient symptoms and co-morbid disease development and mortality. As a result, the traditional AHI is inaccurate and insufficient for use in a diagnostic or therapeutic phase to fully assess and treat a patient's cardiovascular health.

### SUMMARY

Recent studies highlight the importance of nocturnal hypoxemia as the key marker for future cardiovascular events. In this patent application, we disclose a novel visual scanning system that can quantitate hypoxemia burden from standard sleep reports and overnight oximetry reports. Our methodology does not depend on the type of device used to create the oximetry report, as long as the device used meets standard (FDA) medical grade requirements.

An example embodiment as disclosed herein includes a system and method configured for: using a data processor to receive a digitized representation of an oximetry graph of a patient; detecting a level of arterial oxygen saturation (SpO2) in the blood of the patient over a given time period based on the digitized representation of the oximetry graph of the patient; and determining a Hypoxemia Burden Index (HBI) for the patient based on a percentage of time over a unit of measure parameter that the levels of SpO2 in the blood of the patient are below a hypoxemia burden threshold.

### BRIEF DESCRIPTION OF THE DRAWINGS

The various embodiments are illustrated by way of example, and not by way of limitation, in the figures of the accompanying drawings in which:

FIGS. 1 through 3 illustrate examples of a standard oximetry graph generated by sleep testing devices or oximetry software;

FIG. 4 is a processing flow chart illustrating an example embodiment of a method as described herein; and

FIG. 5 shows a diagrammatic representation of machine in the example form of a computer system within which a set of instructions when executed may cause the machine to perform any one or more of the methodologies discussed herein.

### DETAILED DESCRIPTION

In the following description, for purposes of explanation, numerous specific details are set forth in order to provide a thorough understanding of the various embodiments. It will be evident, however, to one of ordinary skill in the art that the various embodiments may be practiced without these specific details.

In the various embodiments described herein, a system and method for image analysis of oximetry burden as a measure of cardiovascular risk are disclosed. In this patent application, we disclose a novel visual scanning system that can quantitate hypoxemia burden from standard sleep reports and overnight oximetry reports or charts. Our methodology does not depend on the type of device used to create the oximetry report, as long as the device used meets standard (FDA) medical grade requirements.

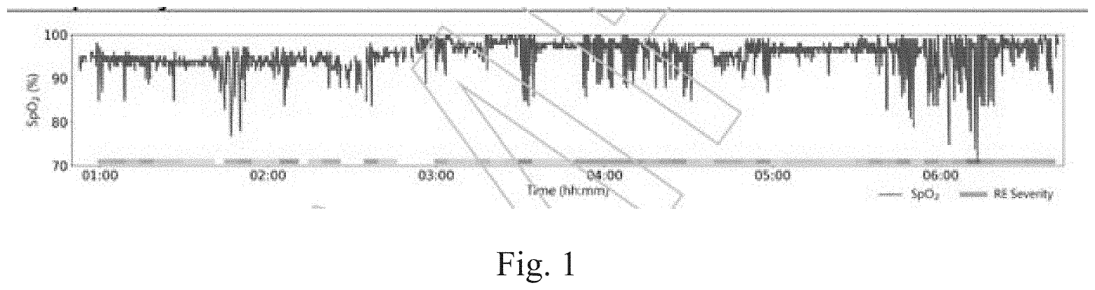

Referring now to FIGS. 1 through 3, examples of a standard oximetry graph generated by sleep testing devices or oximetry software are illustrated. These conventional types of oximetry graphs are visual representations of levels of arterial oxygen saturation (SpO2) in the blood of a patient over a given time period. Because these oximetry graphs are visual representations or images, these images can be scanned and digitized using a standard scanner or scanning device coupled to a computer or computing system. In a particular embodiment, two-dimensional (2D) graph reader software can be used to obtain a digitized representation of the oximetry graph or O2 saturation graph. Using conventional techniques, the digitized representation of the oximetry graph can be transferred as image data to software executing on the computer system. The hypoxemia analysis software executing on the computer system can be programmed and configured to perform the novel hypoxemia analysis operations as described in more detail below. In particular, the hypoxemia analysis software can be configured to perform a method for quantifying a level of hypoxemia burden experienced by a patient over a given time period based on corresponding oximetry graphs of the patient over the same time period.

In an example embodiment, the hypoxemia analysis software can be configured to receive the digitized representation of the oximetry graph of a patient over a given time period. The hypoxemia analysis software can be further configured to detect or determine a level of SpO2 in the blood of the patient over the given time period based on the received digitized representation of the oximetry graph. The hypoxemia analysis software can be further configured with an operator-selectable, operator-set, or operator configurable threshold value or values representing a hypoxemia burden threshold. In general, the hypoxemia burden threshold can represent a level of SpO2 in the blood of the patient at a given point in time. The hypoxemia analysis software can be further configured with an operator-selectable, operator-set, or operator configurable parameter value or values representing a time period or unit of measure over which the levels of SpO2 in the blood of the patient are collected or analyzed. In a particular embodiment, the time period or unit of measure parameter can be one hour. The hypoxemia analysis software can be further configured to use the digitized representation of the oximetry graph of the patient to collect and determine the levels of SpO2 in the blood of the patient over the time period or unit of measure parameter. The hypoxemia analysis software can be further configured to determine a percentage of time over the unit of measure parameter that the levels of SpO2 in the blood of the patient are below the hypoxemia burden threshold.

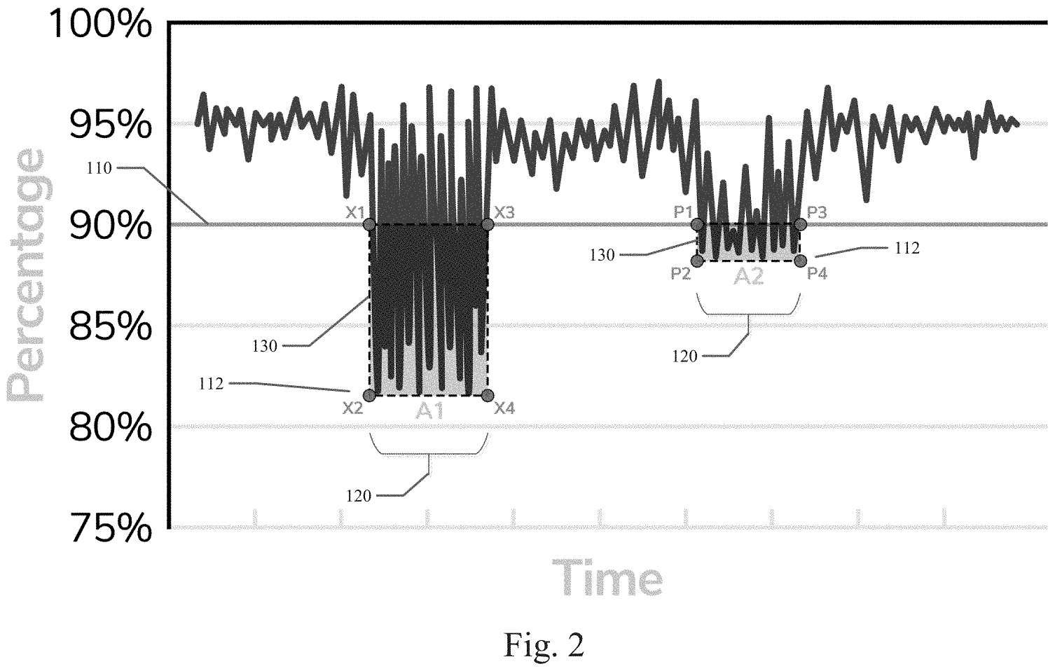

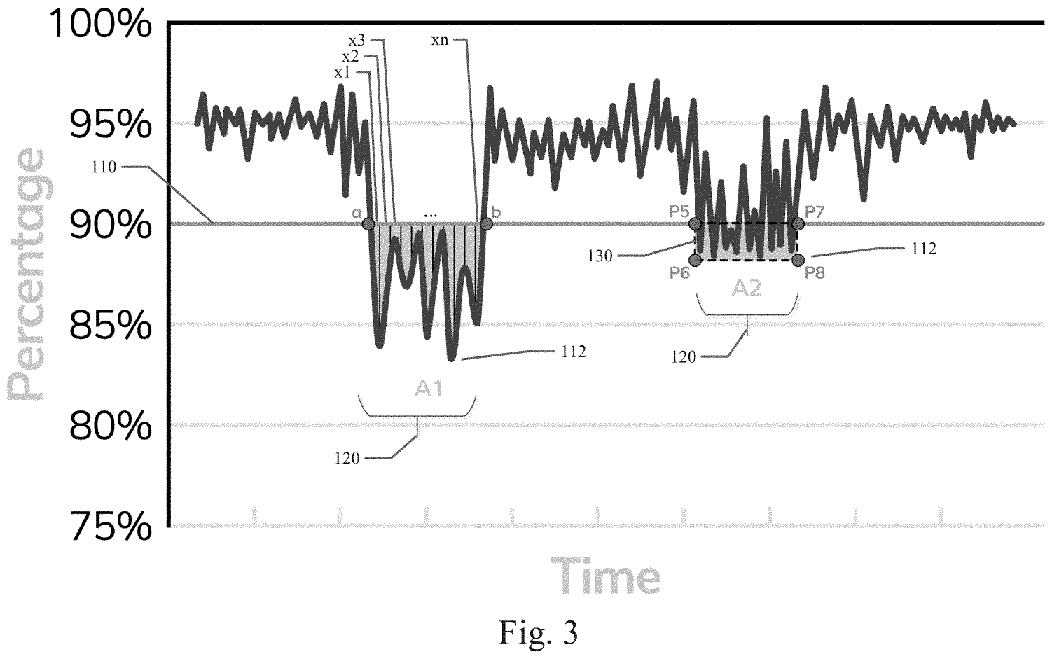

For an example shown in FIGS. 2 and 3, to quantify the amount of hypoxemia of the patient in a given night that is below a 90% level of SpO2 in the blood of the patient, the operator can set the hypoxemia burden threshold 110 at 90%. The operator can also set the unit of measure parameter 120 at one hour. Once an operator or clinician configures the hypoxemia burden threshold 110 and the unit of measure parameter 120 to the desired values, the hypoxemia analysis software can generate bounding boxes 130, which create boundaries around the oximetry graph traces that extend below the hypoxemia burden threshold 110. The bounding boxes 130 can be defined on the y-axis by the boundaries of the hypoxemia burden threshold 110 and a minimal point 112 or a starting point of the traces of the oximetry graph of the patient. The bounding box 130 can be defined on the x-axis by the boundaries of a starting point and an ending point of the time period configured as the unit of measure parameter. The interior of the bounding boxes 130 represent the area of interest relative to the calculation of the hypoxemia of the patient over the pre-defined time period. In particular, the oximetry graph traces that extend into the interior of the bounding boxes 130 represent the hypoxemia burden levels of interest. The hypoxemia analysis software of an example embodiment can be configured to generate a summation of all of the oximetry graph traces that extend into the interior of the bounding boxes 130. In an example embodiment, the hypoxemia analysis software can generate a summation denoted as the Area Under the Curve (AUC). In general, the AUC corresponds to a summation of all of the graph traces at or below the hypoxemia burden threshold 110. The AUC can be divided over the time period configured as the unit of measure parameter to produce a Hypoxemia Burden Index (HBI). As will be apparent to those of ordinary skill in the art in view of the disclosure herein, there are several conventional methods for calculating the area under a curve. For example, the hypoxemia analysis software can generate the AUC using a linear trapezoidal method, a log-linear trapezoidal method, Lagrange polynomial integration, the Purves method, or a pixel raster density method, to name a few. Once the AUC is generated, the AUC can be divided over the time period configured as the unit of measure parameter to produce the HBI. A general formula for the generation of the HBI in an example embodiment is set forth as:

HBI=AUC/CTP
where:
AUC=the Area Under the Curve as described above
CTP=Configured Time Period defined by the unit of measure parameter

For a first particular example of the computation of the HBI in an example embodiment, reference is made again to FIG. 2. As shown in the example of FIG. 2, the oximetry graph traces that extend into the interior of the bounding boxes 130 fit well into the interior of the bounding boxes 130. As a result, the area of these oximetry graph traces can be computed as the area of the bounding boxes 130. As shown in FIG. 2, a first bounding box 130 can be defined by points X1, X2, X3, and X4 and denoted as box A1. A second bounding box 130, of the example shown in FIG. 2, can be defined by points P1, P2, P3, and P4 and denoted as box A2.

Thus, the area of the first bounding box A1 can be calculated as:

Area of box A1={[(X1−X2)+(X3−X4)]*(X3−X1)}/2

The area of the second bounding box A2 can be calculated as:

Area of box A2={[(P1−P2)+(P3−P4)]*(P3−P1)}/2

It will be apparent to those of ordinary skill in the art in view of the disclosure herein that the areas of any number of the bounding boxes of interest can be calculated over the time period of interest for a particular oximetry graph. Once the areas of all bounding boxes of interest are calculated as shown above, the AUC can be calculated as the summation of all of the areas of all bounding boxes of interest. For the example of FIG. 2, the AUC can be calculated as follows:

AUC=(Area of box A1)+(Area of box A2)

Generally, the AUC can be calculated as follows:

AUC=(Area of box A1)+(Area of box A2)+(Area of box An)

Having calculated the AUC, the HBI can be calculated as shown above:

HBI=AUC/CTP

For the example of FIG. 2 and explained above, the HBI can be calculated as follows:

HBI=[(Area of box A1)+(Area of box A2)]/CTP

For a second particular example of the computation of the HBI in an example embodiment, reference is made again to FIG. 3. As shown in the example of FIG. 3, the oximetry graph traces that extend below the hypoxemia burden threshold 110 may not fit well into the interior of a bounding box 130. As a result, the area of these oximetry graph traces cannot be conveniently computed as the area of a bounding box 130. In an alternative computational process as shown in FIG. 3, the oximetry graph traces that extend below the hypoxemia burden threshold 110 and are not rectangular (e.g., area A1 shown in FIG. 3) may be partitioned into segments or time sub-intervals x1, x2, x3, . . . xn from a starting time point a to an ending time point b. The oximetry graph trace A1 in the time interval [a, b] can be further defined using a continuous function. For example, let f(x) be the continuous function on the interval [a,b] as shown in FIG. 3. The oximetry graph trace A1 in interval [a,b] may be partitioned into n segments or time sub-intervals x1, x2, x3, . . . xn, wherein each of the n segments or time sub-intervals x1, x2, x3, . . . xn has a width defined by the following:

Δx=(b−a)/n, Such that a=x0<x1<x2<x3<<xn=b

Given the partitioning of the oximetry graph trace A1 as described above, the area of A1 can be calculated as the integral over a, b as shown below:

$${\int }_{a}^{b} f ( x ) dx \approx {T}_{n} = \frac{Δ x}{2} [ f ( {x}_{0} ) + 2 f ( {x}_{1} ) + 2 f ( {x}_{2} ) + . . . . 2 f ( {x}_{n - 1} ) + f ( {x}_{n} ) ]$$

$$Where , {x}_{i} = a + i Δ x$$

Having computed the integral as set forth above, the area of A1 can be determined between the time points [a,b].

As shown in the example of FIG. 3, there may be other areas or regions of interest of the oximetry graph trace that extend below the hypoxemia burden threshold 110. Another such area or region of interest is area A2 shown in FIG. 3. In this example, area A2 fits well into the interior of the bounding box 130. As a result, the area of A2 can be computed as the area of the bounding box 130 enclosing area A1. As explained above, this area of the box 130 enclosing area A1 can be computed using the following computation:

Area of box A2={[(P1−P2)+(P3−P4)]*(P3−P1)}/2

Having determined the areas of each of the regions of interest in the example oximetry graph shown in FIG. 3, the AUC can be computed as the summation of the areas of each of the regions of interest. This summation is shown below:

AUC=(Area of A1)+(Area of box A2)

Having calculated the AUC, the HBI for the example shown in FIG. 3 can be calculated as explained above and shown below:

HBI=[(Area of A1)+(Area of box A2)]/CTP

The hypoxemia analysis software can also be configured to generate a value denoted as T90, or the percentage of time spent below a 90% oxygen saturation level. As will be apparent to those of ordinary skill in the art in view of the disclosure herein, the 90% level can be configured to a different level of interest. The hypoxemia analysis software can generate a T90 percentage corresponding to a percentage of time the patient's SpO2 is below a 90% level over the time period configured as the unit of measure parameter. A general formula for the generation of the T90 percentage in an example embodiment is set forth as:

T90=AUC/[CTP*(90−SP)]
where:
AUC=the Area Under the Curve as described above
CTP=Configured Time Period defined by the unit of measure parameter
SP=starting point or minimal point 112 of the oximetry graph y-axis

The hypoxemia analysis software can be configured to report the hypoxemia burden threshold and the unit of measure as the “hypoxemia burden per hour of sleep,” denoted as the HBI in order to standardize the amount of hypoxemia burden across recording times. The hypoxemia analysis software and the computer system on which the software is executed provides technology to permit the analysis of HBI on legacy data; because, the hypoxemia analysis software and computer system (e.g., the hypoxemia analysis system) perform the hypoxemia analysis measurements based on the digitized images generated by the oximetry graphs and not based on the raw oximetry sensor data. As a result, the embodiments disclosed herein do not need to access the original raw oximetry sensor data. The HBI generated by the hypoxemia analysis system as disclosed herein can be agnostic to the particular testing device, thus permitting wide distribution and application of the hypoxemia analysis technology. The embodiments disclosed herein provide a solution to speed up the processing of large batches of data, thereby optimizing possible population health prognosticating. Because the detection and quantification of hypoxemia burden in a patient can be an effective measure of the patient's cardiovascular risk, identifying patients with the highest hypoxemia burden may help focus efforts to reduce disease burden and risk of death in this population. Additionally, the hypoxemia analysis system as disclosed herein can be used to validate the HBI as a risk factor for cardiovascular disease, including coronary disease as documented by standard testing (either invasive or noninvasive), acute myocardial infarction, congestive heart failure, and stroke. The hypoxemia analysis system as disclosed herein can quantify the visual, subjective analysis of nocturnal oxygen desaturation severity. The HBI as determined by the hypoxemia analysis system can be compared to the T90, or time spent below 90% oxygen saturation. Because the hypoxemia analysis system as disclosed herein can analyze an area of an oximetry chart (AUC) over time and not just a singular time metric, the HBI generated by the hypoxemia analysis system as disclosed herein can be more clinically significant than the T90 alone. As a result, the hypoxemia analysis system as disclosed herein provides an effective system and method for the detection and quantification of hypoxemia burden in a patient and thus provides an effective tool for measuring the patient's cardiovascular health and risk level.

FIG. 3 is a processing flow chart illustrating an example embodiment of a method 1000 as described herein. The method 1000 of the example embodiment includes: using a data processor to receive a digitized representation of an oximetry graph of a patient (operation 1010); detecting a level of arterial oxygen saturation (SpO2) in the blood of the patient over a given time period based on the digitized representation of the oximetry graph of the patient (operation 1020); and determining a Hypoxemia Burden Index (HBI) for the patient based on a percentage of time over a unit of measure parameter that the levels of SpO2 in the blood of the patient are below a hypoxemia burden threshold (operation 1030).

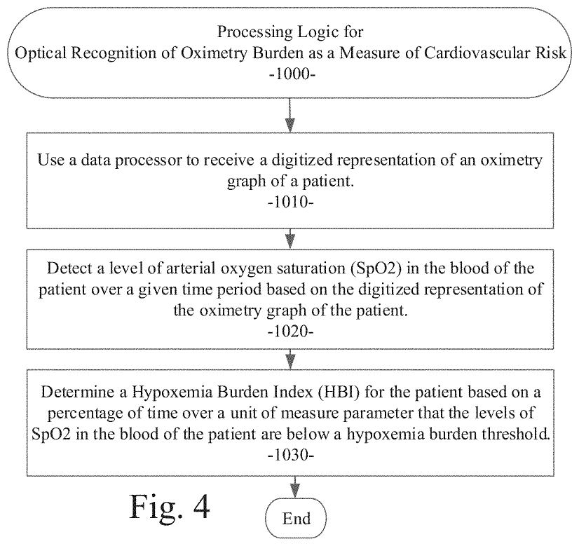

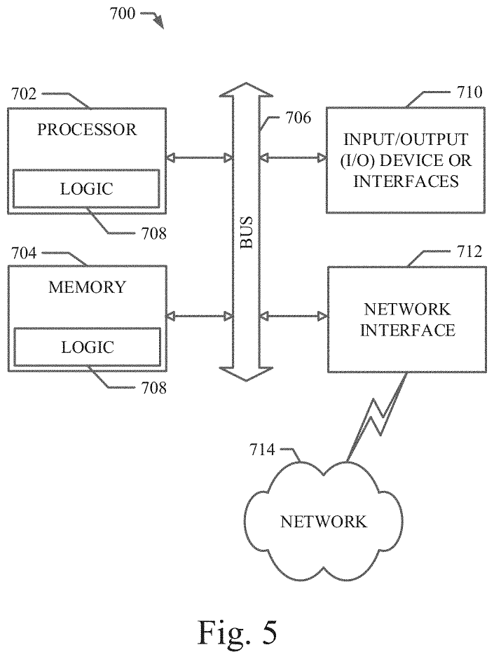

FIG. 4 shows a diagrammatic representation of a machine in the example form of a mobile computing and/or communication system 700 within which a set of instructions when executed and/or processing logic when activated may cause the machine to perform any one or more of the methodologies described and/or claimed herein. In alternative embodiments, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment or as an application running on a virtual server or computational container in a cloud services environment. The machine may be a personal computer (PC), a laptop computer, a tablet computing system, a Personal Digital Assistant (PDA), a cellular telephone, a smartphone, a mobile device, a web appliance, a network router, switch or bridge, a virtual machine running on cloud services, or any machine capable of executing a set of instructions (sequential or otherwise) or activating processing logic that specify actions to be taken by that machine. Further, while only a single machine is illustrated, the term “machine” can also be taken to include any collection of machines that individually or jointly execute a set (or multiple sets) of instructions or processing logic to perform any one or more of the methodologies described and/or claimed herein.

The example mobile computing and/or communication system 700 includes a data processor 702 (e.g., a System-on-a-Chip (SoC), general processing core, graphics core, and optionally other processing logic) and a memory 704, which can communicate with each other via a bus or other data transfer system 706. The mobile computing and/or communication system 700 may further include various input/output (I/O) devices and/or interfaces 710, such as a touchscreen display and optionally a network interface 712. In an example embodiment, the network interface 712 can include one or more radio transceivers configured for compatibility with any one or more standard wireless and/or cellular protocols or access technologies (e.g., 2^{nd} (2G), 2.5, 3^{rd} (3G), 4^{th} (4G), 5^{th} (5G) generation, and future generation radio access for cellular systems, Global System for Mobile communication (GSM), General Packet Radio Services (GPRS), Enhanced Data GSM Environment (EDGE), Wideband Code Division Multiple Access (WCDMA), LTE, CDMA2000, WLAN, Wireless Router (WR) mesh, and the like). Network interface 712 may also be configured for use with various other wired and/or wireless communication protocols, including TCP/IP, UDP, SIP, SMS, RTP, WAP, CDMA, TDMA, UMTS, UWB, WiFi, WiMax, Bluetooth™, IEEE 802.11x, and the like. In essence, network interface 712 may include or support virtually any wired and/or wireless communication mechanisms by which information may travel between the mobile computing and/or communication system 700 and another computing or communication system via network 714.

The memory 704 can represent a machine-readable medium on which is stored one or more sets of instructions, software, firmware, or other processing logic (e.g., logic 708) embodying any one or more of the methodologies or functions described and/or claimed herein. The logic 708, or a portion thereof, may also reside, completely or at least partially within the processor 702 during execution thereof by the mobile computing and/or communication system 700. As such, the memory 704 and the processor 702 may also constitute machine-readable media. The logic 708, or a portion thereof, may also be configured as processing logic or logic, at least a portion of which is partially implemented in hardware. The logic 708, or a portion thereof, may further be transmitted or received over a network 714 via the network interface 712. While the machine-readable medium of an example embodiment can be a single medium, the term “machine-readable medium” should be taken to include a single non-transitory medium or multiple non-transitory media (e.g., a centralized, distributed or cloud based database, and/or associated caches and computing systems) that stores the one or more sets of instructions. The term “machine-readable medium” can also be taken to include any non-transitory medium that is capable of storing, encoding or carrying a set of instructions for execution by the machine and that cause the machine to perform any one or more of the methodologies of the various embodiments, or that is capable of storing, encoding or carrying data structures utilized by or associated with such a set of instructions. The term “machine-readable medium” can accordingly be taken to include, but not be limited to, solid-state memories, optical media, magnetic media, and cloud based virtual memory and virtual storage media.

The various elements of the example embodiments as previously described with reference to the figures may include various hardware elements, software elements, or a combination of both. Examples of hardware elements may include devices, logic devices, components, processors, microprocessors, circuits, processors, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), memory units, logic gates, registers, semiconductor device, chips, microchips, chip sets, and so forth. Examples of software elements may include software components, programs, applications, computer programs, application programs, system programs, software development programs, machine programs, operating system software, middleware, firmware, software modules, routines, subroutines, functions, methods, procedures, software interfaces, application program interfaces (API), web based Software or Platform as a Service's (SaaS or PaaS), instruction sets, computing code, computer code, code segments, computer code segments, words, values, symbols, or any combination thereof. However, determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints, as desired for a given implementation.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus, the following claims are hereby incorporated into the Detailed Description, with each claim standing on its own as a separate embodiment.

What is claimed is:

1. A computer-implemented method comprising:
using a data processor to receive a digitized representation of an oximetry graph of a patient;
detecting a level of arterial oxygen saturation (SpO2) in the blood of the patient over a given time period based on the digitized representation of the oximetry graph of the patient; and
determining a Hypoxemia Burden Index (HBI) for the patient based on a percentage of time over a unit of measure parameter that the levels of SpO2 in the blood of the patient are below a hypoxemia burden threshold.

2. The method of claim 1 wherein the unit of measure parameter and the hypoxemia burden threshold are configurable by an operator.

3. The method of claim 1 wherein the digitized representation of an oximetry graph is image data.

4. The method of claim 1 including scanning images of the oximetry graph of the patient.

5. The method of claim 1 wherein the oximetry graph is a legacy oximetry graph of the patient.

6. The method of claim 1 wherein the oximetry graph of the patient is generated while the patient is asleep.

7. The method of claim 1 including generating a summation of all oximetry graph traces at or below the hypoxemia burden threshold, the summation corresponding to an Area Under the Curve (AUC).

8. The method of claim 7 including dividing the AUC by a time period corresponding to the unit of measure parameter, the result corresponding to the HBI.

9. The method of claim 8 including multiplying the time period corresponding to the unit of measure parameter with a difference between the hypoxemia burden threshold and a starting point or minimal point of the y-axis of the oximetry graph.

10. The method of claim 9 including multiplying the time period corresponding to the unit of measure parameter with a difference between the hypoxemia burden threshold and a starting point or minimal point of the y-axis of the oximetry graph to produce a result, and dividing the AUC by the result to produce a value corresponding to a percentage of time the patient's SpO2 is below a level corresponding to the hypoxemia burden threshold over the time period configured as the unit of measure parameter.

11. The method of claim 1 wherein the HBI is validated as a risk factor for cardiovascular disease, coronary disease, acute myocardial infarction, congestive heart failure, and stroke.

12. A system comprising:
a data processor; and
hypoxemia analysis software executed by the data processor, the hypoxemia analysis software being configured to:
receive a digitized representation of an oximetry graph of a patient;
detect a level of arterial oxygen saturation (SpO2) in the blood of the patient over a given time period based on the digitized representation of the oximetry graph of the patient; and
determine a Hypoxemia Burden Index (HBI) for the patient based on a percentage of time over a unit of measure parameter that the levels of SpO2 in the blood of the patient are below a hypoxemia burden threshold.

13. The system of claim 12 wherein the unit of measure parameter and the hypoxemia burden threshold are configurable by an operator.

14. The system of claim 12 wherein the digitized representation of an oximetry graph is image data.

15. The system of claim 12 being further configured to scan images of the oximetry graph of the patient.

16. The system of claim 12 wherein the oximetry graph of the patient is generated while the patient is asleep.

17. The system of claim 12 being further configured to generate a summation of all oximetry graph traces at or below the hypoxemia burden threshold, the summation corresponding to an Area Under the Curve (AUC).

18. The system of claim 17 being further configured to divide the AUC by a time period corresponding to the unit of measure parameter, the result corresponding to the HBI.

19. The system of claim 18 being further configured to multiply the time period corresponding to the unit of measure parameter with a difference between the hypoxemia burden threshold and a starting point or minimal point of the y-axis of the oximetry graph.

20. The system of claim 19 being further configured to multiply the time period corresponding to the unit of measure parameter with a difference between the hypoxemia burden threshold and a starting point or minimal point of the y-axis of the oximetry graph to produce a result, and divide the AUC by the result to produce a value corresponding to a percentage of time the patient's SpO2 is below a level corresponding to the hypoxemia burden threshold over the time period configured as the unit of measure parameter.
